(19) United States
(12) Patent Application Publication
(10) Pub. No.: US 2009/0312280 A1
(43) Pub. Date: Dec. 17, 2009

(54) COMBINATION THERAPY OF (2R,Z)-2-AMINO-2-CYCLOHEXYL-N-(5-(1-METHYL-1H-PYRAZOL-4-YL)-1-OXO-2,6-DIHYDRO-1H-[1,2]DIAZEPINO[4,5,6-CD]INDOL-8-YL)ACETAMIDE

(75) Inventors: Kenna Anderes, San Diego, CA (US); Alessandra Blasina, San Diego, CA (US)

Correspondence Address: PFIZER INC, 10555 SCIENCE CENTER DRIVE, SAN DIEGO, CA, 92121, US

(73) Assignee: Pfizer Inc.

(21) Appl. No.: 12/295,986

(22) PCT Filed: Mar. 26, 2007
(86) PCT No.: PCT/IB07/00928

§ 371 Date: Mar. 17, 2009

Related U.S. Application Data
(60) Provisional application No. 60/789,276, filed on Apr. 4, 2006.

(51) Int. Cl. A61K 31/551; A61K 31/7068; A61P 35/00

(52) U.S. Cl. 514/49; 514/220

(57) ABSTRACT

The present invention relates to novel combination therapies of (2R,Z)-2-amino-2-cyclohexyl-N-(5-(1-methyl-1H-pyrazol-4-yl)-1-oxo-2,6-dihydro -1H-[1,2]diazepino[4,5,6-cd]indol-8-yl)acetamide (compound 1), a pharmaceutically acceptable salt thereof, or a mixture thereof, in combination with an anti-cancer agent or radiation therapy.

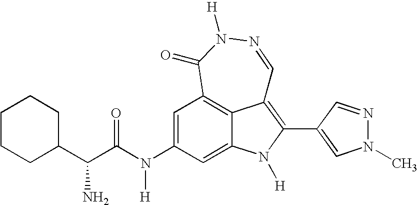

This application is the national stage filing under 35 U.S.C. 371, of Patent Cooperation Treaty Application No. PCT/IB2007/00928, filed Mar. 26, 2007, which claims the benefit of U.S. Provisional Patent Application Nos. 60/789,276 filed Apr. 4, 2006 and 60/887,299 filed Jan. 30, 2007, the disclosures of which are herein incorporated by reference in their entireties.

### FIELD OF THE INVENTION

The present invention is directed to methods of using (2R,Z)-2-amino-2-cyclohexyl-N-(5-(1-methyl-1H-pyrazol-4-yl)-1-oxo-2,6-dihydro-1H-[1,2]diazepino[4,5,6-cd]indol-8-yl) acetamide or a pharmaceutically acceptable salt thereof, in combination with an anti-cancer agent or radiation therapy to treat cancer in a mammal.

### BACKGROUND OF THE INVENTION

The compound (2R,Z)-2-amino-2-cyclohexyl-N-(5-(1-methyl-1H-pyrazol-4-yl)-1-oxo-2,6-dihydro-1H-[1,2]diazepino[4,5,6-cd]indol-8-yl)acetamide (also referred to as “Compound 1”),

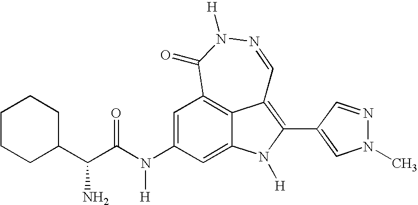

1 as well as pharmaceutically acceptable salts thereof, is described in U.S. Pat. No. 6,967,198, issued Nov. 22, 2005, the disclosure of which is incorporated herein by reference.

Many anticancer agents, as well as radiation therapy, cause DNA damage to cells, especially cancer cells. CHK1 inhibition enhances the anti-cancer effect of these anti-cancer agents or radiation therapy by abrogating the S and G₂ arrest of those DNA damaged cells and thus leading to mitotic catastrophe and cell death of these cells. (2R,Z)-2-amino-2-cyclohexyl-N-(5-(1-methyl-1H-pyrazol-4-yl)-1-oxo-2,6-dihydro-1H-[1,2]diazepino[4,5,6-cd]indol-8-yl)acetamide is a potent CHK1 protein kinase inhibitor. Use of (2R,Z)-2-amino-2-cyclohexyl-N-(5-(1-methyl-1H-pyrazol-4-yl)-1-oxo-2,6-dihydro-1H[1,2]diazepino[4,5,6-cd]indol-8-yl)acetamide, a pharmaceutically acceptable salt or solvate thereof, or a mixture thereof, in combination with an anti-cancer agent or radiation therapy will greatly enhance the anti-cancer effect of the anti-cancer agent or radiation therapy.

### SUMMARY OF THE INVENTION

In one embodiment, the invention provides a method of treating a hyperproliferative disorder in a mammal comprising administering to the mammal a therapeutically effective amount of (2R,Z)-2-amino-2-cyclohexyl-N-(5-(1-methyl-1H-pyrazol-4-yl)-1-oxo-2,6-dihydro-1H-[1,2]diazepino[4,5,6-cd]indol-8-yl)acetamide, a pharmaceutically acceptable salt or solvate thereof, or a mixture thereof, in combination with a therapeutically effective amount of an anti-hyperproliferative treatment selected from an anti-hyperproliferative agent and radiation therapy.

In one particular aspect of this embodiment, the anti-hyperproliferative agent is selected from inhibitors of the enzyme farnesyl protein transferase and inhibitors of the receptor tyrosine kinase PDGFr. Preferably, the anti-hyperproliferative agent is a compound disclosed and claimed in the following: U.S. Pat. No. 6,080,769; U.S. Pat. No. 6,194,438; U.S. Pat. No. 6,258,824; U.S. Pat. No. 6,586447; U.S. Pat. No. 6,071,935; U.S. Pat. No. 6,495,564; and U.S. Pat. No. 6,150,377; U.S. Pat. No. 6,596,735; U.S. Pat. No. 6,479,513; WO 01/40217; U.S. 2003-0166675. Each of the foregoing patents and patent applications is herein incorporated by reference in their entirety.

In one particular aspect of this embodiment, the anti-hyperproliferative agent is a PDGRr inhibitor. The PDGRr inhibitor includes but is not limited to those disclosed in international patent application publication numbers WO01/40217 and WO2004/020431, the contents of which are incorporated in their entirety for all purposes. Preferred PDGFr inhibitors include Pfizer's CP-673,451 and CP-868,596 and its salts.

In another embodiment, the invention provides a method of treating cancer in a mammal comprising administering to the mammal a therapeutically effective amount of (2R,Z)-2-amino-2-cyclohexyl-N-(5-(1-methyl-1H-pyrazol-4-yl)-1-oxo-2,6-dihydro-1H-[1,2]diazepino[4,5,6-cd]indol-8-yl)acetamide, a pharmaceutically acceptable salt or solvate thereof, or a mixture thereof, in combination with a therapeutically effective amount of an anti-cancer treatment selected from an anti-cancer agent and radiation therapy.

In one particular aspect of this embodiment, and in combination with any other particular aspects not inconsistent, the method enhances the therapeutic effect of the anti-cancer treatment.

In another particular aspect of this embodiment, and in combination with any other particular aspects not inconsistent, the method shows a synergistic therapeutic effect of (2R,Z)-2-amino-2-cyclohexyl-N-(5-(1-methyl-1H-pyrazol-4-yl)-1-oxo-2,6-dihydro-1H-[1,2]diazepino[4,5,6-cd]indol-8-yl)acetamide, a pharmaceutically acceptable salt or solvate thereof, or a mixture thereof, and the anti-cancer treatment.

In another particular aspect of this embodiment, and in combination with any other particular aspects not inconsistent, the cancer is selected from colon cancer, prostate cancer, breast cancer and leukemia. Even more preferably, the cancer is colon cancer.

In another particular aspect of this embodiment, and in combination with any other particular aspects not inconsistent, the anti-cancer treatment is an anti-cancer agent. Preferably, the anti-cancer agent is a chemical or biological substance which is clinically shown to treat cancer. More preferably, the anti-cancer agent is selected from the group consisting of actinomycin D, adriamycin, amsacrine, ara-C, 9-(3-D-arabinosyl-2-fluoroadenine, BCNU, bleomycin, camptothecin, carboplatin, 2-chloro-2-deoxyadenosine, CPT-11, cyclophosphamide, docetaxel, doxorubicin, edotecarin, etoposide, fludarabine, 5-fluorouracil (5-FU), gemcitabine, HU-Gemzar, Irinotecan, methotrexate, 6-Mpurine, mytomicin-C, paclitaxel, cis-platin, SN-38, taxol, thiotepa, 6-thioguanine, trimetrexate vinblastine, vincristine, and VP-16. Even more preferably, the anti-cancer agent is selected from the group consisting of gemcitabine, irinotecan, docetaxel, SN-38, carboplatin, doxorubicin and mytomicin C. Even more preferably, the anti-cancer agent is gemcitabine. Even more preferably, the anti-cancer agent is irinotecan. Even more preferably, the anti-cancer agent is docetaxel.

In another particular aspect of this embodiment, and in combination with any other particular aspects not inconsistent, the anti-cancer agent is a DNA damaging agent. Preferably, the “DNA damaging agent” is a chemical or biological substance that is clinically shown to treat cancer. More preferably, the DNA damaging agent is selected from the group consisting of alkylating agents, antimetabolites, antitumor antibiotics, platinum analogs, topoisomerase I inhibitors and topoisomerase II inhibitors.

In another particular aspect of this embodiment, and in combination with any other particular aspects not inconsistent, the anti-cancer agent is an alkylating agent. Preferably, the alkylating agent is selected from the group consisting of apaziquone, altretamine, brostallicin, bendamustine, busulfan, carboquone, carmustine, chlorambucil, chlormethine, cyclophosphamide, estramustine, fotemustine, glufosfamide, ifosfamide, lomustine, mafosfamide, mechlorethamine oxide, mecillinam, melphalan, mitobronitol, mitolactol, nimustine, nitrogen mustard N-oxide, pipobroman, ranimustine, temozolomide, thiotepa, treosulfan, and trofosframide. Even more preferably, the alkylating agent is cyclophosphamide.

In another particular aspect of this embodiment, and in combination with any other particular aspects not inconsistent, the anti-cancer agent is an antimetabolite. Preferably, the antimetabolite is selected from the group consisting of Alimta, Ara-C, 5-azacitidine, capecitabine, carmofur, cladribine, clofarabine, cytarabine, cytosine arabinoside, decitabine, disodium premetrexed, doxifluridine, eflornithine, enocitabine, ethynylcytidine, floxuridine, fludarabine, 5-fluorouracil (5-FU), gemcitabine, hydroxyurea, leucovorin, melphalan, 6-mercaptopurine, methotrexate, mitoxantrone, 6-Mpurine, pentostatin, pelitrexol, raltitrexed, riboside, methotrexate, mercaptopurine, nelarabine, nolatrexed, ocfosfate, tegafur, 6-thioguanine (6-TG), tioguanine, triapine, trimetrexate, vidarabine, vincristine, vinorelbine and UFT. More preferably, the antimetabolite is selected from 5-fluorouracil and gemcitabine. Even more preferably, the antimetabolite is gemcitabine.

In another particular aspect of this embodiment, and in combination with any other particular aspects not inconsistent, the anti-cancer agent is an antitumor antibiotic. Preferably, the antitumor antibiotic is selected from the group consisting of aclarubicin, actinomycin D, amrubicin, annamycin, adriamycin, bleomycin, dactinomycin, daunorubicin, doxorubicin, elsamitrucin, epirubicin, galarubicin, idarubicin, mitomycin C, mycophenolic acid, nemorubicin, neocarzinostatin, pentostatin, peplomycin, pirarubicin, rebeccamycin, stimalamer, streptozocin, valrubicin and zinostatin. More preferably, the antibiotic is selected from the group consisting of actinomycin D, bleomycin, doxorubicin and mitomycin-C. Even more preferably, the antitumor antibiotic is selected from mitomycin-C and doxorubicin.

In another particular aspect of this embodiment, and in combination with any other particular aspects not inconsistent, the anti-cancer agent is a platinum analogue. Preferably, the platinum analogue is selected from the group consisting of carboplatin (Paraplatin), cisplatin, Eloxatin (oxaliplatin, Sanofi) eptaplatin, lobaplatin, nedaplatin and satrplatin. More preferably, the platinum analog is selected from cisplatin, carboplatin and Eloxatin (oxaliplatin). Even more preferably, the platinum analog is carboplatin.

In another particular aspect of this embodiment, and in combination with any other particular aspects not inconsistent, the anti-cancer agent is a topoisomerase I inhibitor. Preferably, the topoisomerase I inhibitor is selected from the group consisting of BN-80915 (Roche), camptothecin, CPT-11, edotecarin, exatecan, irinotecan, orathecin (Supergen), SN-38, and topotecan. More preferably, the topoisomerase I inhibitor is selected from irinotecan, SN-38 and topotecan. Even more preferably, the topoisomerase I inhibitor is irinotecan.

In another particular aspect of this embodiment, and in combination with any other particular aspects not inconsistent, the anti-cancer agent is a topoisomerase II inhibitor. Preferably, the toposimerase II inhibitor is selected from amsacrine, etoposide, etoposide phosphate and epirubicin (Ellence).

In another particular aspect of this embodiment, and in combination with any other particular aspects not inconsistent, the anti-cancer agent includes one or more agents selected from the group consisting of aclarubicn, amonafide, belotecan, camptothecin, 10-hydroxycamptothecin, 9-aminocamptothecin, diflomotecan, irinotecan HCl (Camptosar), edotecarin, epirubicin (Ellence), etoposide, exatecan, gimatecan, lurtotecan, mitoxantrone, pirarubicin, pixantrone, rubitecan, sobuzoxane, SN-38, tafluposide, topotecan. Preferably, the anti-cancer agent includes one or more agents selected from the group consisting of camptothecin, 10-hydroxycamptothecin, 9-aminocamptothecin, irinotecan HCl (Camptosar), edotecarin, epirubicin (Ellence), etoposide, SN-38, topotecan, and combinations thereof.

In another particular aspect of this embodiment, and in combination with any other particular aspects not inconsistent, the anti-cancer agent is a mitotic inhibitor. Preferably, the mitotic inhibitor is selected from the group consisting of docetaxel (Taxotere), estramustine, paclitaxel, razoxane, taxol, teniposide, vinblastine, vincristine, vindesine and vinorelbine. More preferably, the mitotic inhibitor is selected from docetaxel, vincristine, vinblastine and taxol. Even more preferably, the mitotic inhibitor is docetaxel.

In another particular aspect of this embodiment, and in combination with any other particular aspects not inconsistent, the anti-cancer treatment is radiation therapy.

In another particular aspect of this embodiment, and in combination with any other particular aspects not inconsistent, at least one dose, preferably at least 20% of all doses, more preferably at least 50% of all doses, even more preferably at least 90% of all doses, even more preferably each single dose, of (2R,Z)-2-amino-2-cyclohexyl-N-(5-(1-methyl-1H-pyrazol-4-yl)-1-oxo-2,6-dihydro-1H-[1,2]diazepino[4,5,6-cd]indol-8-yl)acetamide, a pharmaceutically acceptable salt or solvate thereof, or a mixture thereof, is administered 1 to 48 hours, more preferably 2 to 40 hours, more preferably 4 to 32 hours, more preferably 8 to 28 hours, even more preferably 16 to 26 hours, even more preferably 23 to 25 hours, even more preferably, about 24 hours after a dose of the anti-cancer treatment is administered.

In another particular aspect of this embodiment, and in combination with any other particular aspects not inconsistent, at least one dose, preferably at least 20% of all doses, more preferably at least 50% of all doses, even more preferably at least 90% of all doses, even more preferably each single does, of (2R,Z)-2-amino-2-cyclohexyl-N-(5-(1-methyl-1H-pyrazol-4-yl)-1-oxo-2,6-dihydro-1H-[1,2]diazepino[4,5,6-cd]indol-8-yl)acetamide, a pharmaceutically acceptable salt or solvate thereof, or a mixture thereof, is administered simultaneously with a dose of the anti-cancer treatment. “Simultaneously” used herein refers to within 4 hours, preferably within 2 hours, preferably within 1 hour, even more preferably within 30 minutes, 15 minutes or 5 minutes, before or after.

In another particular aspect of this embodiment, and in combination of any other particular aspects not inconsistent, the method selectively targets p53-defective cells while having minimal cytotoxic effects on normal (p53-competent) cells.

In another particular aspect of this embodiment, and in combination of any other particular aspects not inconsistent, the anti-cancer agent is an anti-angiogenesis agent. Preferably, the anti-angiogenesis agent is selected from EGF inhibitors, EGFR inhibitors, VEGF inhibitors, VEGFR inhibitors, TIE2 inhibitors, IGF1R inhibitors, COX-II (cyclooxygenase II) inhibitors, MMP-2 (matrix-metalloprotienase 2) inhibitors, and MMP-9 (matrix-metalloprotienase 9) inhibitors.

Preferred VEGF inhibitors, include for example, Avastin (bevacizumab), an anti-VEGF monoclonal antibody of Genentech, Inc. of South San Francisco, Calif. Additional VEGF inhibitors include CP-547,632 (Pfizer Inc., NY, USA), AG13736 (Pfizer Inc.), ZD-6474 (AstraZeneca), AEE788 (Novartis), AZD-2171, VEGF Trap (Regeneron/Aventis), Vatalanib (also known as PTK-787, ZK-222584: Novartis & Schering AG), Macugen (pegaptanib octasodium, NX-1838, EYE-001, Pfizer Inc./Gilead/Eyetech), IM862 (Cytran Inc. of Kirkland, Washington, USA); and angiozyme, a synthetic ribozyme from Ribozyme (Boulder, Colo.) and Chiron (Emeryville, Calif.) and combinations thereof. VEGF inhibitors useful in the practice of the present invention are described in U.S. Pat. Nos. 6,534,524 and 6,235,764, both of which are incorporated in their entirety for all purposes. Additional VEGF inhibitors are described in, for example in WO 99/24440, in WO 95/21613, WO 99/61422, U.S. Pat. No. 5,834,504, WO 98/50356, U.S. Pat. No. 5,883,113 U.S. Pat. No. 5,886,020, U.S. Pat. No. 5,792,783, U.S. Pat. No. 6,653,308, WO 99/10349, WO 97/32856, WO 97/22596, WO 98/54093, WO 98/02438, WO 99/16755, and WO 98/02437, all of which are herein incorporated by reference in their entirety.

Preferred EGRF inhibitors include, but are not limited to Iressa (gefitinib, AstraZeneca), Tarceva (erlotinib or OSI-774, OSI Pharmaceuticals Inc.), Erbitux (cetuximab, Imclone Pharmaceuticals, Inc.), EMD-7200 (Merck AG), ABX-EGF (Amgen Inc. and Abgenix Inc.), HR3 (Cuban Government), IgA antibodies (University of Erlangen-Nuremberg), TP-38 (IVAX), EGFR fusion protein, EGF-vaccine, anti-EGFr immunoliposomes (Hermes Biosciences Inc.) and combinations thereof. Even more preferably, the EGFR inhibitor is selected from Iressa, Erbitux, Tarceva and combinations thereof.

Other anti-angiogenic agent include acitretin, fenretinide, thalidomide, zoledronic acid, angiostatin, aplidine, cilengtide, combretastatin A-4, endostatin, halofuginone, rebimastat, removab, Revlimid, squalamine, ukrain, Vitaxin and combinations thereof.

In another particular aspect of this embodiment, and in combination of any other particular aspects not inconsistent, the anti-cancer agent is a pan kinase inhibitor. Preferred pan kinase inhibitors include Sutent™ (sunitinib), described in U.S. Pat. No. 6,573,293 (Pfizer, Inc, NY, USA).

In another particular aspect of this embodiment, and in combination of any other particular aspects not inconsistent, anti-cancer agent is selected from from pan Erb receptor inhibitors or ErbB2 receptor inhibitors, such as CP-724,714 (Pfizer, Inc.), CI-1033 (canertinib, Pfizer, Inc.), Herceptin (trastuzumab, Genentech Inc.), Omitarg (2C4, pertuzumab, Genentech Inc.), TAK-165 (Takeda), GW-572016 (Ionafarnib, GlaxoSmithKline), GW-282974 (GlaxoSmithKline), EKB-569 (Wyeth), PKI-166 (Novartis), dHER2 (HER2 Vaccine, Corixa and GlaxoSmithKline), APC8024 (HER2 Vaccine, Dendreon), anti-HER2/neu bispecific antibody (Decof Cancer Center), B7.her2.IgG3 (Agensys), AS HER2 (Research Institute for Rad Biology & Medicine), trifunctional bispecific antibodies (University of Munich) and mAB AR-209 (Aronex Pharmaceuticals Inc) and mAB 2B-1 (Chiron) and combinations thereof.

In another particular aspect of this embodiment, and in combination of any other particular aspects not inconsistent, the anti-cancer agent is selected from Pfizer's MEK1/2 inhibitor PD325901, Array Biopharm's MEK inhibitor ARRY-142886, Bristol Myers' CDK2 inhibitor BMS-387,032, Pfizer's CDK inhibitor PD0332991 and AstraZeneca's AXD-5438, and combinations thereof.

In another particular aspect of this embodiment, and in combination of any other particular aspects not inconsistent, anti-cancer agent is selected from celecoxib (U.S. Pat. No. 5,466,823), valdecoxib (U.S. Pat. No. 5,633,272), parecoxib (U.S. Pat. No. 5,932,598), deracoxib (U.S. Pat. No. 5,521,207), SD-8381 (U.S. Pat. No. 6,034,256, Example 175), ABT-963 (WO 2002/24719), rofecoxib (CAS No. 162011-90-7), MK-663 (or etoricoxib) as disclosed in WO 1998/03484, COX-189 (Lumiracoxib) as disclosed in WO 1999/11605, BMS-347070 (U.S. Pat. No. 6,180,651), NS-398 (CAS 123653-11-2), RS 57067 (CAS 17932-91-3), 4-Methyl-2-(3,4-dimethylphenyl)-1-(4-sulfamoyl-phenyl)-1H-pyrrole, 2-(4-Ethoxyphenyl)-4-methyl-1-(4-sulfamoylphenyl)-1H-pyrrole, and meloxicam.

In another particular aspect of this embodiment, and in combination of any other particular aspects not inconsistent, the anti-cancer agent is selected from Genasense (augmerosen, Genta), Panitumumab (Abgenix/Amgen), Zevalin (Schering), Bexxar (Corixa/GlaxoSmithKline), Abarelix, Alimta, EPO 906 (Novartis), discodermolide (XAA-296), ABT-510 (Abbott), Neovastat (Aeterna), enzastaurin (Eli Lilly), Combrestatin A4P (Oxigene), ZD-6126 (AstraZeneca), flavopiridol (Aventis), CYC-202 (Cyclacel), AVE-8062 (Aventis), DMXAA (Roche/Antisoma), Thymitaq (Eximias), Temodar (temozolomide, Schering Plough) and Revilimd (Celegene) and combinations thereof.

In another particular aspect of this embodiment, and in combination of any other particular aspects not inconsistent, the anti-cancer agent is selected from CyPat (cyproterone acetate), Histerelin (histrelin acetate), Plenaixis (abarelix depot), Atrasentan (ABT-627), Satraplatin (JM-216), thalomid (Thalidomide), Theratope, Temilifene (DPPE), ABI-007 (paclitaxel), Evista (raloxifene), Atamestane (Biomed-777), Xyotax (polyglutamate paclitaxel), Targetin (bexarotine) and combinations thereof.

In another particular aspect of this embodiment, and in combination of any other particular aspects not inconsistent, the anti-cancer agent is selected from Trizaone (tirapazamine), Aposyn (exisulind), Nevastat (AE-941), Ceplene (histamine dihydrochloride), Orathecin (rubitecan), Virulizin, Gastrimmune (G17DT), DX-8951f (exatecan mesylate), Onconase (ranpirnase), BEC2 (mitumoab), Xcytrin (motexafin gadolinium) and combinations thereof.

In another particular aspect of this embodiment, and in combination of any other particular aspects not inconsistent, the anti-cancer agent is selected from CeaVac (CEA), NeuTrexin (trimetresate glucuronate) and combinations thereof. Additional anti-tumor agents may be selected from the following agents, OvaRex (oregovomab), Osidem (IDM-1), and combinations thereof. Additional anti-tumor agents may be selected from the following agents, Advexin (ING 201), Tirazone (tirapazamine), and combinations thereof. Additional anti-tumor agents may be selected from the following agents, RSR13 (efaproxiral), Cotara (131I chTNT 1/b), NBI-3001 (IL-4) and combinations thereof. Additional anti-tumor agents may be selected from the following agents, Canvaxin, GMK vaccine, PEG Interon A, Taxoprexin (DHA/paciltaxel), and combinations thereof.

The terms “alkylating agents”, “antimetabolites”, “antitumor antibiotics”, “platinum analogs”, “topoisomerase I inhibitors”, “topoisomerase II inhibitors” and “mototic inhibitors” used herein, refer to the classes of clinically used anti-cancer agent, chemical or biological. Each of these terms includes any of the current clinically used anti-cancer agents that falls within the particular class, as well as any future clinical anti-cancer agent not yet invented but will fall into the particular class. For examples of each of these classes of anti-cancer agent, see Physician's Cancer Chemotherapy Drug Manual, 2006, ISBN 0-7637-4019-5. For more comprehensive lists of each of these classes of anti-cancer agent, see Martindale's Complete Drug Reference, 34^{th} Edition.

The term “anti-cancer treatment” refers to an “anti-cancer agent” or “radiation therapy”, as defined herein.

The term “anti-cancer agent” refers to any substances, chemical or biological, that can be used to treat cancer.

The term “DNA damaging agent” refers to any anti-cancer agent, chemical or biological that directly or indirectly prevents the normal replication or normal function of DNA in a mammal. Examples of “DNA damaging agent” includes, but are not limited to alkylating agents, antimetabolite, anti-cancer antibiotics, platimum analogs, topoisomerase I inhibitors and topoisomerase II inhibitors, as defined herein.

The term “in combination with” refers to the relative timing of the administration of a therapeutic treatment, such as (2R,Z)-2-amino-2-cyclohexyl-N-(5-(1-methyl-1H-pyrazol-4-yl)-1-oxo-2,6-dihydro-1H-[1,2]diazepino[4,5,6-cd]indol-8-yl)acetamide, a pharmaceutically acceptable salt or solvate thereof, or a mixture thereof, to a mammal in need thereof, to that of another therapeutic treatment, such as an anti-cancer agent or radiation therapy, the relative timing being those normally used in the field of medicine for combination therapy. In particular, relative timing can be sequential or simultaneous. A preferable embodiment of sequential administration is administering the anti-cancer agent or radiation therapy first followed by the administering (2R,Z)-2-amino-2-cyclohexyl-N-(5-(1-methyl-1H-pyrazol-4-yl)-1-oxo-2,6-dihydro-1H-[1,2]diazepino[4,5,6-cd]indol-8-yl)acetamide, a pharmaceutically acceptable salt or solvate thereof, or a mixture thereof, within 24 hours.

The term “hyperproliferative disorder” refers to abnormal cell growth that is independent of normal regulatory mechanisms (e.g., loss of contact inhibition), including the abnormal growth of normal cells and the growth of abnormal cells. This includes, but is not limited to, the abnormal growth of tumor cells (tumors), both benign and malignant. Examples of such benign proliferative diseases are psoriasis, benign prostatic hypertrophy, human papilloma virus (HPV), and restinosis.

The term “cancer” includes, but is not limited to, lung cancer, bone cancer, pancreatic cancer, skin cancer, cancer of the head or neck, cutaneous or intraocular melanoma, uterine cancer, ovarian cancer, rectal cancer, cancer of the anal region, stomach cancer, colon cancer, breast cancer, uterine cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, chronic or acute leukemia, lymphocytic lymphomas, cancer of the bladder, cancer of the kidney or ureter, renal cell carcinoma, carcinoma of the renal pelvis, cancers of the central nervous system (CNS), primary CNS lymphoma, spinal axis tumors, brain stem glioma, pituitary adenoma, or a combination of one or more of the foregoing cancers. In another embodiment of said method, said abnormal cell growth is a benign proliferative disease, including, but not limited to, psoriasis, benign prostatic hypertrophy or restinosis.

The term “mediated by CHK1 protein kinase activity” refers to biological or molecular processes that are regulated, modulated, or inhibited by CHK1 protein kinase activity.

The term “pharmaceutically acceptable salt(s)”, refers to salts of acidic or basic groups which may be present in a compound. Compounds that are basic in nature are capable of forming a wide variety of salts with various inorganic and organic acids. The acids that may be used to prepare pharmaceutically acceptable acid addition salts of such basic compounds are those that form non-toxic acid addition salts, i.e., salts containing pharmacologically acceptable anions, such as the acetate, benzenesulfonate, benzoate, bicarbonate, bisulfate, bitartrate, borate, bromide, calcium edetate, camsylate, carbonate, chloride, clavulanate, citrate, dihydrochloride, edetate, edislyate, estolate, esylate, ethylsuccinate, fumarate, gluceptate, gluconate, glutamate, glycollylarsanilate, hexylresorcinate, hydrabamine, hydrobromide, hydrochloride, iodide, isothionate, lactate, lactobionate, laurate, malate, maleate, mandelate, mesylate, methylsulfate, mucate, napsylate, nitrate, oleate, oxalate, pamoate (embonate), palmitate, pantothenate, phospate/diphosphate, polygalacturonate, salicylate, stearate, subacetate, succinate, tannate, tartrate, teoclate, tosylate, triethiodode, and valerate salts. Preferably preferred salts include phosphate and gluconate salts.

The term “pharmaceutical composition” refers to a mixture of one or more of the compounds described herein, or physiologically/pharmaceutically acceptable salts or solvates thereof, with other chemical components, such as physiologically/pharmaceutically acceptable carriers and excipients. The purpose of a pharmaceutical composition is to facilitate administration of a compound to an organism.

The term “radiation therapy” refers to medical use of radiation to control malignant cells.

The term “therapeutically effective amount” generally refers to an amount of a compound, a pharmaceutically acceptable salt or solvate thereof, or a mixture thereof, being administered which will relieve to some extent one or more of the symptoms of the disorder being treated. In particular, when the term is used in describing a combination therapy, “therapeutically effective amount” refers to the amount of a particular therapeutic which will 1) enhance the therapeutic effect of another therapeutic such as an anti-cancer agent or radiation therapy, or 2) in combination with the other therapeutic, relieve to some extent one or more of the symptoms of the disorder being treated. In reference to the treatment of cancer, relieving symptoms of the disease being treated includes a) reducing the size of the tumor; b) inhibiting (that is, slowing to some extent, preferably stopping) tumor metastasis; and c) inhibiting to some extent (that is, slowing to some extent, preferably stopping) tumor growth.

The term “treating”, as used herein, unless otherwise indicated, means reversing, alleviating, inhibiting the progress of, or preventing the disorder or condition to which such term applies, or one or more symptoms of such disorder or condition. The term “treatment”, as used herein, unless otherwise indicated, refers to the act of treating as “treating” is defined immediately above.

### DETAILED DESCRIPTION OF THE INVENTION

As used in this section only, the term “compound 1” refers to (2R,Z)-2-amino-2-cyclohexyl-N-(5-(1-methyl-1H-pyrazol-4-yl)-1-oxo-2,6-dihydro-1H-[1,2]diazepino[4,5,6-cd]indol-8-yl)acetamide, a pharmaceutically acceptable salt or solvate thereof, or a mixture thereof; “MTD” refers to maximum tolerated dose; Q3d×4 refers a dosing schedule of once every 3 days for 4 treatment; Q1w×3 refers to a dosing schedule of once every week for 3 treatment.

(2R,Z)-2-amino-2-cyclohexyl-N-(5-(1-methyl-1H-pyrazol-4-yl)-1-oxo-2,6-dihydro-1H-[1,2]diazepino[4,5,6-cd]indol-8-yl)acetamide has been studied in a variety of in vitro and in vivo systems to determine potency against its molecular target, kinase selectivity, mechanism of action, PK/PD relationship, and chemopotentiation of antitumor efficacy.

### I. Kinase Selectivity

(2R,Z)-2-amino-2-cyclohexyl-N-(5-(1-methyl-1H-pyrazol-4-yl)-1-oxo-2,6-dihydro-1H-[1,2]diazepino[4,5,6-cd]indol-8-yl)acetamide is a potent, ATP-competitive inhibitor of CHK1. The Ki value of (2R,Z)-2-amino-2-cyclohexyl-N-(5-(1-methyl-1H-pyrazol-4-yl)-1-oxo-2,6-dihydro-1H-[1,2]diazepino[4,5,6-cd]indol-8-yl)acetamide against CHK1 (1-289) catalytic domain was 0.49±0.29 nM.

Kinase selectivity of (2R,Z)-2-amino-2-cyclohexyl-N-(5-(1-methyl-1H-pyrazol-4-yl)-1-oxo-2,6-dihydro-1H-[1,2]diazepino[4,5,6-cd]indol-8-yl)acetamide relative to Chk1 was evaluated in biochemical kinase screening assays against a panel of over 100 protein kinases. Eight kinases showed a ratio of the IC₅₀ or Ki of the kinase being screened over the Kᵢ of CHK1 catalytic domain, of less or about 100 fold. These eight kinases are Aurora-A, FGFR3, Flt3, Fms (CSF1R), Ret, VEGFR2, Yes and CHK2. (Table 1) Kinases that are most pharmacologically relevant to a CHK1 inhibitor for selectivity considerations are those for which transient intermittent inhibition would influence cell cycle progression (eg, CDK's, mitotic kinases), checkpoint control (eg, CHK2, ATM, ATR), or act on apoptotic pathways (eg, AKT, p38). Based on this, VEGFR2, Fms/CSF1R, FGFR2, Flt3, and Ret are not considered to be relevant because sustained inhibition is required to evoke observable pharmacology from these RTK's. Similarly, no effect is expected from transient inhibition of Yes kinase, as the Yes knockout mouse exhibits no significant phenotype. Aurora-A is a relevant kinase, but it has been found that the enzyme assay does not correlate well with cell activity. In a cell-based functional assay, (2R,Z)-2-amino-2-cyclohexyl-N-(5-(1-methyl-1H-pyrazol-4-yl)-1-oxo-2,6-dihydro-1H-[1,2]diazepino[4,5,6-cd]indol-8-yl)acetamide showed over 100-fold selectivity against Aurora kinase. Finally, the selectivity ratio over CHK2 is essentially equal to 100-fold, and we have observed no evidence that CHK2 activity is modulated by (2R,Z)-2-amino-2-cyclohexyl-N-(5-(1-methyl-1H-pyrazol-4-yl)-1-oxo-2,6-dihydro-1H-[1,2]diazepino[4,5,6-cd]indol-8-yl)acetamide in cell-based or ex vivo assays. Table 1 shows the IC₅₀ or Kᵢ value of (2R,Z)-2-amino-2-cyclohexyl-N-(5-(1-methyl-1H-pyrazol-4-yl)-1-oxo-2,6-dihydro-1H-[1,2]diazepino[4,5,6-cd]indol-8-yl)acetamide against selected kinases and the ratio between the IC₅₀ or Kᵢ of the selected kinase over Ki of CHK1.

TABLE 1
IC₅₀ Against Selected Kinases

| Kinase | IC₅₀ (nM) | Fold Selectivity |
|---|---|---|
| EGFR2 | 8 (Kᵢ) | 16 |
| Yes | 14^{a} | 29 |
| Fms | 10 | 20 |
| Aurora-A | 23 | 47 |
| FGFR3 | 23 | 47 |
| Flt3 | 25 | 51 |
| Ret | 39^{a} | 80 |
| Chk2 | 47 (Kᵢ) | 96 |
| P70S6K | 61 | 124 |
| Rsk1 | 98 | 200 |
| Axl | 117 | 239 |
| Fgr | 153 | 312 |
| Rsk3 | 171 | 349 |
| Bmx | 233 | 476 |
| Lyn | 266 | 543 |
| PAR-1Ba | 350 | 714 |
| Blk(m) | 365 | 745 |
| Lck | 396 | 808 |
| PDK1 | 439 | 896 |
| cSRC | 442 | 902 |
| Rsk2 | 621 | 1267 |
| Abl(m) | 929 | 1896 |
| Fyn | 953 | 1945 |
| TrkA | 1270 | 2592 |
| PRK2 | 1980 | 4041 |
| PDGFRa | 2810 | 5735 |
| PKBa | 9200 | 18776 |
| EphB4 | >10000 | >20000 |

### II. Cytotoxicity Enhancing Effect in Cell Based Functional Assays.

Checkpoint-mediated cell cycle arrest is a typical response to DNA damage induced by chemotherapy agents or radiation. In combination with commonly used chemotherapy agents like gemcitabine, irinotecan, and doxorubicin, (2R,Z)-2-amino-2-cyclohexyl-N-(5-(1-methyl-1H-pyrazol-4-yl)-1-oxo-2,6-dihydro-1H-[1,2]diazepino[4,5,6-cd]indol-8-yl)acetamide abrogates the S and G₂ checkpoints induced by DNA damaging agents and enhances cytotoxicity. This checkpoint abrogating activity and enhanced cytotoxic activity shows selectivity for p53-defective cancer cell lines over p53-competent normal cells. Checkpoint abrogation is characterized by threonine-14 and tyrosine-15 dephosphorylation and activation of the mitotic protein kinase CDK1, premature mitosis, mitotic catastrophe, and ultimately apoptotic cell death. A series of experiments were performed to 1) demonstrate abrogation of DNA damage induced cell cycle checkpoint; 2) evaluate chempotentiating activity of (2R,Z)-2-amino-2-cyclohexyl-N-(5-(1-methyl-1H-pyrazol-4-yl)-1-oxo-2,6-dihydro-1H-[1,2]diazepino[4,5,6-cd]indol-8-yl)acetamide in combination with some chemotherapeutic agents; and 3) demonstrate selectivity for p53-deficient cancer cells.

Checkpoint Abrogating Activity: The Histone H3 phosphorylation assay detects cells entering mitosis and represents the primary in vitro cell-based assay used to measure the cellular potency of (2R,Z)-2-amino-2-cyclohexyl-N-(5-(1-methyl-1H-pyrazol-4-yl)-1-oxo-2,6-dihydro-1H-[1,2]diazepino[4,5,6-cd]indol-8-yl)acetamide in abrogating the G₂ checkpoint induced by camptothecin. The EC₅₀ value was 45 nM, as measured by an increase in Histone H3 phosphorylation on Ser10, a marker of entry into mitosis. In the absence of DNA damage, (2R,Z)-2-amino-2-cyclohexyl-N-(5-(1-methyl-1H-pyrazol-4-yl)-1-oxo-2,6-dihydro-1H-[1,2]diazepino[4,5,6-cd]indol-8-yl)acetamide had no effect on cell cycle. Upon combination with gemcitabine, flow cytometry analysis shows abrogation of gemcitabine induced S-Phase arrest with (2R,Z)-2-amino-2-cyclohexyl-N-(5-(1-methyl-1H-pyrazol-4-yl)-1-oxo-2,6-dihydro-1H-[1,2]diazepino[4,5,6-cd]indol-8-yl)acetamide. The time-dependent decrease in the S-Phase cells induced by (2R,Z)-2-amino-2-cyclohexyl-N-(5-(1-methyl-1H-pyrazol-4-yl)-1-oxo-2,6-dihydro-1H-[1,2]diazepino[4,5,6-cd]indol-8-yl)acetamide corresponded to an increase in the G₂-M and G₀₋G₁ cell populations, demonstrating that cells are entering mitosis and attempting to re-enter the cell cycle. Flow cytometry analysis confirmed a significant increase in apoptotic cells in the gemcitabine and (2R,Z)-2-amino-2-cyclohexyl-N-(5-(1-methyl-1H-pyrazol-4-yl)-1-oxo-2,6-dihydro-1H-[1,2]diazepino[4,5,6-cd]indol-8-yl)acetamide combination treatment compared with the gemcitabine treatment alone.

Chemopotentiation: Cell survival and MTT (3-(4,5-dimethylthiazol-2-yl)-2,5-diphenyltetrazolium bromide assay) assays were performed in a panel of p53-defective human cancer cell lines to characterize the activity of (2R,Z)-2-amino-2-cyclohexyl-N-(5-(1-methyl-1H-pyrazol-4-yl)-1-oxo-2,6-dihydro-1H-[1,2]diazepino[4,5,6-cd]indol-8-yl)acetamide in enhancing the cytotoxic effect of gemcitabine, irinotecan, carboplatin, doxorubicin, and mitomycin C. (2R,Z)-2-amino-2-cyclohexyl-N-(5-(1-methyl-1H-pyrazol-4-yl)-1-oxo-2,6-dihydro-1H-[1,2]diazepino[4,5,6-cd]indol-8-yl)acetamide alone caused no significant effect on cell viability compared with control (untreated) cells. In combination with gemcitabine, ((2R,Z)-2-amino-2-cyclohexyl-N-(5-(1-methyl-1H-pyrazol-4-yl)-1-oxo-2,6-dihydro-1H-[1,2]diazepino[4,5,6-cd]indol-8-yl)acetamide induced significant potentiation (89%) of gemcitabine cytotoxicity compared with gemcitabine alone. (2R,Z)-2-amino-2-cyclohexyl-N-(5-(1-methyl-1H-pyrazol-4-yl)-1-oxo-2,6-dihydro-1H-[1,2]diazepino[4,5,6-cd]indol-8-yl)acetamide induced robust and consistent potentiation with most agents, with some variability observed between cell lines (Table 2). In Table 2, Gemcitabine was used at a concentration that induces no or minimal toxicity (<10%) in the absence of (2R,Z)-2-amino-2-cyclohexyl-N-(5-(1-methyl-1H-pyrazol-4-yl)-1-oxo-2,6-dihydro-1H-[1,2]diazepino[4,5,6-cd]indol-8-yl)acetamide: 5 nM (Colo205 cells), 10 nM (MDA-MB-231, HT29, and K562 cells) or 20 nM (PC-3 cells).

TABLE 2
In Vitro Combination Cytotoxicity in Selected Cell Lines

| | Cell line (Tumor type) | | | | |
|---|---|---|---|---|---|
| | | | | MDA- | |
| | HT29 | Colo205 | PC-3 | MB-231 | K562 |
| | (Colon) | (Colon) | (Prostate) | (Breast) | (Leukemia) |
| IC₅₀ (μM)^{a} | 1.8 | 1.3 | 1.6 | 1.4 | 0.42 |
| OTSI^{b} | 8.5 | 14 | 13.2 | 2.1 | 9.3 |
| DNA damaging | PF₅₀^{c} | | | | |
| agent | | | | | |
| Gemcitabine | 9 | 11.3 | 12.2 | 3.6 | 5.6 |
| SN-38 | 3.7 | 2.1 | 1.3 | 2.4 | 1.9 |
| Carboplatin | 3 | 5.4 | 3.1 | 2.55 | 1.9 |
| Doxorubicin | 2.2 | 1.1 | 1.5 | 2.25 | 1.1 |
| Mytomicin C | 3.7 | 5.3 | ND^{d} | 1.2 | ND^{c} |

^{a}(2R,Z)-2-amino-2-cyclohexyl-N-(5-(1-methyl-1H-pyrazol-4-yl)-1-oxo-2,6-dihydro-1H-[1,2]diazepino[4,5,6-cd]indol-8-yl)acetamide was used in absence of another cytotoxic agent.
^{b}OTSI (On-Target Selectivity Index) was calculated as IC₅₀ of (2R,Z)-2-amino-2-cyclohexyl-N-(5-(1-methyl-1H-pyrazol-4-yl)-1-oxo-2,6-dihydro-1H-[1,2]diazepino[4,5,6-cd]indol-8-yl)acetamide) over IC₅₀ of combination treatment.
^{c}PF₅₀ (Potentiation Factor₅₀) was calculated as IC_{50,(cytotoxic agent alone)}/IC_{50,(combination treatment)}. (2R,Z)-2-amino-2-cyclohexyl-N-(5-(1-methyl-1H-pyrazol-4-yl)-1-oxo-2,6-dihydro-1H-[1,2]diazepino[4,5,6-cd]indol-8-yl)acetamide was used at 8x EC₅₀ (360 nM) in all the cell lines, except for K-562 cells, where it was used at 4x EC₅₀ (180 nM).
^{d}Not determined; in these assays the curves' profile did not allow calculation of an accurate PF₅₀.

Selectivity for p53-Defective Cells: (2R,Z)-2-amino-2-cyclohexyl-N-(5-(1-methyl-1H-pyrazol-4-yl)-1-oxo-2,6-dihydro-1H-[1,2]diazepino[4,5,6-cd]indol-8-yl)acetamide in combination with DNA directed chemotherapy is expected to selectively target p53-defective cancer cells while having minimal cytotoxic effects on normal (p53-competent) cells. In order to assess the cytotoxic effect of (2R,Z)-2-amino-2-cyclohexyl-N-(5-(1-methyl-1H-pyrazol-4-yl)-1-oxo-2,6-dihydro-1H-[1,2]diazepino[4,5,6-cd]indol-8-yl)acetamide in combination with chemotherapy agents in normal cells, a cell survival assay was performed in HUVEC cells. (2R,Z)-2-amino-2-cyclohexyl-N-(5-(1-methyl-1H-pyrazol-4-yl)-1-oxo-2,6-dihydro-1H-[1,2]diazepino[4,5,6-cd]indol-8-yl)acetamide was used in combination with either gemcitabine or camptothecin, both used at a fixed concentration that induces minimal cell toxicity (<10%). The highest concentration (12×EC₅₀, 540 nM) of (2R,Z)-2-amino-2-cyclohexyl-N-(5-(1-methyl-1H-pyrazol-4-yl)-1-oxo-2,6-dihydro-1H-[1,2]diazepino[4,5,6-cd]indol-8-yl)acetamide in combination causes 31.2% or 21.7 increase in cell kill compared with gemcitabine or camptothecin alone, respectively. The cytotoxic effect induced by the combination treatment in HUVEC cells is negligible compared with the cytotoxicity induced by the same treatment in tumor cells. The minimal toxicity induced by (2R,Z)-2-amino-2-cyclohexyl-N-(5-(1-methyl-1H-pyrazol-4-yl)-1-oxo-2,6-dihydro-1H-[1,2]diazepino[4,5,6-cd]indol-8-yl)acetamide in p53-competent non-tumor cells in combination with chemotherapy provides evidence to support its selectivity for p53-defective cancer cells and potential to have minimal adverse effects in normal cells. A cell survival assay was also performed in HTC116 cells (human colon carcinoma) that were transiently transfected with a plasmid containing either p53 wild type or mutant. In the mutant p53 HCT116 cells the combination of (2R,Z)-2-amino-2-cyclohexyl-N-(5-(1-methyl-1H-pyrazol-4-yl)-1-oxo-2,6-dihydro-1H-[1,2]diazepino[4,5,6-cd]indol-8-yl)acetamide and gemcitabine induced 44% cell growth inhibition compared with gemcitabine alone, whereas in the wild type p53 HCT116 cells, the same combination treatment induced only 15% cell growth inhibition compared with gemcitabine alone. These results confirm that p53-defective cancer cells are more vulnerable to Chk1 inhibition than their p53-competent counterparts.

### III. Chemopotentiation Effect in In Vivo Studies

Combination study of (2R,Z)-2-amino-2-cyclohexyl-N-(5-(1-methyl-1H-pyrazol-4-yl)-1-oxo-2,6-dihydro-1H-[1,2]diazepino[4,5,6-cd]indol-8-yl)acetamide with a cytotoxic agent were performed in HT29 and Colo205 human colon carcinoma xenografts. Experiments 1 to 39 were conducted in mice xenografts. Experiments 40-42 were conducted in rat xenografts. Specifically, irinotecan combination studies were conducted in HT29 and Colo205. Gemcitabine combination studies were conducted in Colo205. Docetaxel combination studies were conducted in Colo205. The chemopotentiation of (2R,Z)-2-amino-2-cyclohexyl-N-(5-(1-methyl-1H-pyrazol-4-yl)-1-oxo-2,6-dihydro-1H-[1,2]diazepino[4,5,6-cd]indol-8-yl)acetamide was demonstrated in all the above combination studies.

Gemcitabine and irinotecan are DNA directed cytotoxics known to induce checkpoint activation and subsequent S/G₂M-phase arrest. (2R,Z)-2-amino-2-cyclohexyl-N-(5-(1-methyl-1H-pyrazol-4-yl)-1-oxo-2,6-dihydro-1H-[1,2]diazepino[4,5,6-cd]indol-8-yl)acetamide was generally administered 24 hours after the previous does of Gemcitabine or Irinotecan. Docetaxel is an antimitotics where recent discoveries describe a novel function for CHK1 in the mitotic checkpoint. (2R,Z)-2-amino-2-cyclohexyl-N-(5-(1-methyl-1H-pyrazol-4-yl)-1-oxo-2,6-dihydro-1H-[1,2]diazepino[4,5,6-cd]indol-8-yl)acetamide was administered simultaneously with Docetaxel. In each of these studies, (2R,Z)-2-amino-2-cyclohexyl-N-(5-(1-methyl-1H-pyrazol-4-yl)-1-oxo-2,6-dihydro-1H-[1,2]diazepino[4,5,6-cd]indol-8-yl)acetamide was administered in sodium acetate and 4% dextrose/water solution at 5 mL/kg. The results are summarized in Table 3.

TABLE 3
In Vivo Chemopotentiation of Antitumor Activity

| | | | | | | | Growth | |
|---|---|---|---|---|---|---|---|---|
| Exp | tumor | | Amt A | Amt B | | % poten | delay | |
| No. | type | cytotoxic agent | (mg/kg) | (mg/kg) | % TGI | TGI | (days) | % TTP |
| 1 | Colo205 | Gemcitabine | 120 | 0 | 58 | n/a | 8 | n/a |
| 2 | Colo205 | Gemcitabine | 120 | 4 | 58 | | 8 | |
| 3 | Colo205 | Gemcitabine | 120 | 8 | 63 | 12 | 8 | |
| 4 | Colo205 | Gemcitabine | 120 | 12 | 68 | 23 | 8.5 | |
| 5 | Colo205 | Gemcitabine | 120 | 20 | 76 | 43 | 12 | 17 |
| 6 | Colo205 | Gemcitabine | 120 | 24 | 81 | 55 | 12 | 17 |
| 7 | Colo205 | Gemcitabine | 120 | 40 | 90 | 75 | 20 | 50 |
| 8 | Colo205 | Irinotecan | 10 | 0 | 35 | | 18 | |
| 9 | Colo205 | Irinotecan | 10 | 4 | 50 | 22 | 18 | |
| 10 | Colo205 | Irinotecan | 10 | 20 | 69 | 52 | 24 | 33 |
| 11 | Colo205 | Irinotecan | 10 | 40 | 59 | 37 | 20.5 | 12 |
| 12 | Colo205 | Irinotecan | 10 | 0 | 35 | | 18 | |
| 13 | Colo205 | Irinotecan | 30 | 0 | 65 | | 22 | |
| 14 | Colo205 | Irinotecan | 60 | 0 | 83 | | 48 | |
| 15 | Colo205 | Irinotecan | 10 | 40 | 59 | 37 | 20.5 | 12 |
| 16 | Colo205 | Irinotecan | 30 | 40 | 73 | 24 | 34 | 54 |
| 17 | Colo205 | Irinotecan | 60 | 40 | 91 | 47 | 52 | 8 |
| 18 | Colo205 | Docetaxel | 30 | 30 | 85 | | 54 | |
| 19 | Colo205 | Docetaxel | 30 | 7.5 | 90 | 35 | 53 | |
| 20 | Colo205 | Docetaxel | 30 | 15 | 93 | 57 | 68 | 36 |
| 21 | Colo205 | Docetaxel | 30 | 30 | 103 | 122 | 74 | 50 |
| 22 | Colo205 | Docetaxel | 15 | 15 | 49 | | 4.75 | |
| 23 | Colo205 | Docetaxel | 15 | 15 | 65 | 31 | 19.75 | 72 |
| 24 | Colo205 | Docetaxel | 15 | 30 | 79 | 59 | 41.5 | 177 |
| 25 | Colo205 | Docetaxel | 15 | 60 | 99 | 99 | 35 | 145 |
| 26 | HT29 | Irinotecan | 10 | 0 | 26 | | 1.75 | |
| 27 | HT29 | Irinotecan | 10 | 4 | 26 | | 7.5 | 29 |
| 28 | HT29 | Irinotecan | 10 | 20 | 61 | 47 | 9.5 | 39 |
| 29 | HT29 | Irinotecan | 10 | 40 | 57 | 42 | 10.5 | 44 |
| 30 | HT29 | Irinotecan | 10 | 0 | 8 | | 2.75 | |
| 31 | HT29 | Irinotecan | 30 | 0 | 13 | | 4 | |
| 32 | HT29 | Irinotecan | 60 | 0 | 49 | | 12.5 | |
| 33 | HT29 | Irinotecan | 10 | 40 | 22 | 15 | 1.0 | 0 |
| 34 | HT29 | Irinotecan | 30 | 40 | 66 | 60 | 17.5 | 61 |
| 35 | HT29 | Irinotecan | 60 | 40 | 77 | 55 | 21 | 28 |
| 36 | HT29 | Irinotecan | 50 | 0 | 65 | | 7.5 | |
| 37 | HT29 | Irinotecan | 100 | 0 | 70 | | 11.5 | |
| 38 | HT29 | Irinotecan | 50 | 40 | 77 | 36 | 14 | 24 |
| 39 | HT29 | Irinotecan | 100 | 40 | 89 | 62 | 21 | 49 |
| 40 | HT29 | Irinotecan | 100 | 0 | 45 | | 4.0 | |
| 41 | HT29 | Irinotecan | 100 | 25 | 58 | 23 | 4.0 | |
| 42 | HT29 | Irinotecan | 100 | 100 | 77 | 58 | 8.0 | 24 |

Used in Table 3, “Exp No.” refers to Example No; “Amt A” refers to the amount of the cytotoxic agent being administered to the xenograft per does; “Amt B” refers to the amount of (2R,Z)-2-amino-2-cyclohexyl-N-(5-(1-methyl-1H-pyrazol-4-yl)-1-oxo-2,6-dihydro-1H-[1,2]diazepino[4,5,6-cd]indol-8-yl)acetamide being administered to the xenograft per dose; % TGI (tumor growth inhibition) was calculated as 100×[1−(TV_{f}−Tvᵢ)_{Treated}/(TV_{f}−Tvᵢ)_{Vehicle}], where TV_{f} and Tvᵢ are the final dose+2 days and initial average tumor volume of a group respectively; % Potentiated TGI was calculated as 100×[1−(TV_{f}−Tvᵢ)_{Combination}/(TV_{f}−Tvᵢ)_{Cytotoxic alone}], where TV_{f} and Tvᵢ are the final dose+2 days and initial average tumor volume of a group respectively; growth delay was calculated as Treatment−Vehicle (T−C) for median days to reach 2 doublings (800 mm³); % TTP ER (time to progression enhancement ratio) was calculated as Delay [(combination)/Delay (cytotoxic alone)×100−100)].

In Exp No. 1 to 17, Irinotecan or gemcitabine, where applicable, was administered intraperitoneal (IP) according to Q3d×4, (2R,Z)-2-amino-2-cyclohexyl-N-(5-(1-methyl-1H-pyrazol-4-yl)-1-oxo-2,6-dihydro-1H-[1,2]diazepino[4,5,6-cd]indol-8-yl)acetamide was administered IP according to Q3d×4 beginning 24 hours after irinotecan or gemcitabine.

In Exp No. 18 to 25, Docetaxel and (2R,Z)-2-amino-2-cyclohexyl-N-(5-(1-methyl-1H-pyrazol-4-yl)-1-oxo-2,6-dihydro-1H-[1,2]diazepino[4,5,6-cd]indol-8-yl)acetamide were administered administered intraperitoneal (IP) simultaneously according to a Q1w×3 schedule. In Exp. 25, (2R,Z)-2-amino-2-cyclohexyl-N-(5-(1-methyl-1H-pyrazol-4-yl)-1-oxo-2,6-dihydro-1H-[1,2]diazepino[4,5,6-cd]indol-8-yl)acetamide was administered for two cycles for a total dose of 120 mg/kg.

In Exp No. 26 to 35, Irinotecan was administered intraperitoneal (IP) according to Q3d×4, and (2R,Z)-2-amino-2-cyclohexyl-N-(5-(1-methyl-1H-pyrazol-4-yl)-1-oxo-2,6-dihydro-1H-[1,2]diazepino[4,5,6-cd]indol-8-yl)acetamide was administered IP according to Q3d×4 beginning 24 hours after irinotecan or gemcitabine.

In Exp No. 36-39, Irinotecan was administered IP Q1w×3, and (2R,Z)-2-amino-2-cyclohexyl-N-(5-(1-methyl-1H-pyrazol-4-yl)-1-oxo-2,6-dihydro-1H-[1,2]diazepino[4,5,6-cd]indol-8-yl)acetamide was administered IP twice weekly, 24 and 72 hours after administration of Irinotecan, for three weeks.

In Exp No. 40 to 42, Irinotecan was administered IP according to Q3d×4, and (2R,Z)-2-amino-2-cyclohexyl-N-(5-(1-methyl-1H-pyrazol-4-yl)-1-oxo-2,6-dihydro-1H-[1,2]diazepino[4,5,6-cd]indol-8-yl)acetamide was administered via two hour IV infusion according to Q3d×4 beginning 24 hours after administration of Irinotecan.

MTD of (2R,Z)-2-amino-2-cyclohexyl-N-(5-(1-methyl-1H-pyrazol-4-yl)-1-oxo-2,6-dihydro-1H-[1,2]diazepino[4,5,6-cd]indol-8-yl)acetamide was decided to be 40 mg/kg Q3d×4 or 60 mg/kg Q1w×3 assessed by the occurrence of mean body weight loss of 10% to 20%.

IV. In vivo Studies of the Radio Sensitizing effect of Compound 1

Female Balb/c nude mice (Age 6 weeks) were inoculated on the right hind limb with 3×10⁶ A431 cells in PBS and allowed the tumor to grow to a mean tumor volume ˜100 mm³. The mice were randomized into groups of 10 animals each group.

The unanaesthetized mice were then subjected to radiation. Radiation was delivered using a 6 MeV high dose rate electron beam from a Varian 2100 Linear Accelerator (Palo Alto, Calif.). The dose rate used was 20 Gy/min. The depth-dose characteristics of the electron beam were such that dose uniformity to within ±5 % was obtained over a 10 mm depth of tissue. This was sufficient to cover all tumor irradiated. The tumor was irradiated though a 25 mm square collimator cut from 3 mm thick lead sheet attached to a 6 mm thick Perspex sheet. The separation between the tumor and the lower (Perspex) side of the surface collimator was approximately 25 mm. The apparatus was supported on a plate heated to 37° C. in order to reduce the effects of heat loss in the mice. Radiation doses were calculated and delivered by a senior radiation physicist. Radiotherapy was given as described above on Days 0-4 as 2, 3 or 4 Gy daily fractions.

(2R,Z)-2-amino-2-cyclohexyl-N-(5-(1-methyl-1H-pyrazol-4-yl)-1-oxo-2,6-dihydro-1H-[1,2]diazepino[4,5,6-cd]indol-8-yl)acetamide (Compound 1) was prepared as a pH buttered aqueous solution. The solution was prepared immediately prior to dosing and (15 mg/kg) was administered by intraperitoneal injection at 15 mg/kg on Days 0-4 immediately following radiotherapy. The above solution that contains no Compound 1 is considered as the “drug vehicle” or “vehicle”. Drug vehicle was administered at 0.1 ml/10 g body weight on the same schedule.

Each group of mice were treated with Compound 1 only, radiation only, or the combination of Compound 1 and radiation. Animals were sacrificed when Tumor Volume Ratio (TVR) reached or exceeded 4 or if the mouse had lost more than 15% of its baseline body weight at Day 0. Tumor Volume Ratio is defined as the ratio between the tumors volume at a particular time and the baseline tumor volume, which is the tumor volume at Day 0.

Tumor volume was measured three times weekly from day 0 to day 11 and even further to day 23. Tumor volume was measured using electronic calipers and calculated as length/2×width². The mean tumor volume was calculated for each group of mice. Table 4 shows the mean tumor volume of each group of mice that were not treated, treated with drug vehicle, Compound 1, radiation or the combination of Compound 1 and radiation.

TABLE 4
Antitumor Efficacy of Radiation Therapy in Combination with Compound
1 in A431 Mice Xenographs

| Time | No | Vehicle | Cmpd | 2 Gy + | 3 Gy + | 4 Gy + | 2 Gy + | 3 Gy + | 4 Gy + |
|---|---|---|---|---|---|---|---|---|---|
| (days) | treatment | only | 1 only | Vehicle | vehicle | Vehicle | Cmpd 1 | Cmpd 1 | Cmpd 1 |
| 0 | 104.78 | 104.36 | 104.23 | 101.97 | 104.63 | 105.32 | 94.51 | 104.26 | 101.97 |
| 2 | 166.76 | 149.37 | 158.07 | 150.81 | 161.82 | 155.29 | 145.53 | 149.88 | 150.81 |
| 4 | 218.14 | 216.64 | 242.46 | 209.44 | 219.40 | 209.07 | 198.57 | 210.23 | 209.44 |
| 7 | 342.07 | 302.86 | 418.28 | 307.93 | 279.82 | 259.44 | 255.23 | 200.48 | 307.93 |
| 9 | 460.18 | 391.63 | 580.35 | 294.63 | 232.98 | 242.81 | 339.76 | 164.45 | 294.63 |
| 11 | 814.89 | 792.78 | 790.82 | 487.78 | 342.32 | 368.51 | 401.96 | 210.35 | 487.78 |
| 14 | | | | 815.01 | 364.79 | 484.81 | 644.80 | 283.51 | 815.01 |
| 16 | | | | | 528.51 | 643.06 | | 408.01 | |
| 18 | | | | | | 889.01 | | 433.83 | |
| 21 | | | | | | | | 395.17 | |
| 23 | | | | | | | | 513.31 | |

Table 5 shows the Tumor Growth Delay and the Enhancement ratio, base on the tumor volume data shown in Table 4. Tumor growth delay is defined as the median time in days for tumors to reach a TVR of 4 minus time for vehicle control tumors to reach the same size. Normalized growth delay is defined as the time in days for tumors in combination treated mice to reach TVR of 4 minus time in days for tumors in drug alone treated mice to reach the same size. Enhancement ratio is defined as normalized tumor growth delay in mice treated with drug and radiation divided by tumor growth delay in mice treated with radiation alone.

TABLE 5
In vivo study of the Radio sensitizing effect
of Compound 1 in A431 Tumor Xenographs

| | | | Normalized | |
|---|---|---|---|---|
| | Median | Tumor | Tumor | |
| | time to | Growth | Growth | Enhance- |
| | TVR = 4 | Delay | Delay | ment |
| | (days) | (days) | (days) | Ratio |
| Compound 1 alone | 7.35 | −1.05 | | |
| Radiation (2 Gy × 5) | 10.7 | 2.3 | | |
| Radiation (3 Gy × 5) | 11.3 | 2.9 | | |
| Radiation (4 Gy × 5) | 11.9 | 3.5 | | |
| Compound 1 plus | 11.1 | 2.7 | 3.75 | 1.6 |
| Radiation (2 Gy × 5) | | | | |
| Compound 1 plus | 15.1 | 6.7 | 7.75 | 2.7 |
| Radiation (3 Gy × 5) | | | | |
| Compound 1 plus | 13 | 4.6 | 5.65 | 1.6 |
| Radiation (4 Gy × 5) | | | | |

1-16. (canceled)

17. A method of treating a mammalian cancer comprising administering to a mammal in need a therapeutically effective amount of (2R,Z)-2-amino-2-cyclohexyl-N-(5-(1-methyl-1H-pyrazol-4-yl)-1-oxo-2,6-dihydro-1H-[1,2]diazepino[4,5,6-cd]indol-8-yl)acetamide, a pharmaceutically acceptable salt or solvate thereof, or a mixture thereof, in combination with therapeutically effective amount of an anti-cancer treatment selected from an anti-cancer agent and a radiation therapy.

18. The method of claim 17, wherein administering of (2R,Z)-2-amino-2-cyclohexyl-N-(5-(1-methyl-1H-pyrazol-4-yl)-1-oxo-2,6-dihydro-1H-[1,2]diazepino[4,5,6-cd]indol-8-yl)acetamide, a pharmaceutically acceptable salt or solvate thereof, or a mixture thereof, enhances the therapeutic effect of the anti-cancer treatment.

19. The method of claim 17, wherein the method shows a synergistic therapeutic effect of administering separately (2R,Z)-2-amino-2-cyclohexyl-N-(5-(1-methyl-1H-pyrazol-4-yl)-1-oxo-2,6-dihydro-1H-[1,2]diazepino[4,5,6-cd]indol-8-yl)acetamide, a pharmaceutically acceptable salt or solvate thereof, or a mixture thereof, and the anti-cancer treatment.

20. The method of claim 17, wherein the cancer is selected from colon cancer, prostate cancer, breast cancer and leukemia.

21. The method of claim 20, wherein the cancer is colon cancer.

22. The method of claim 17, wherein the anti-cancer treatment is a therapeutically effective amount of an anti-cancer agent.

23. The method of claim 22, wherein the anti-cancer agent is selected from the group consisting of actinomycin D, adriamycin, amsacrine, ara-C, 9-(3-D-arabinosyl-2-fluoroadenine, BCNU, bleomycin, camptothecin, carboplatin, 2-chloro-2-deoxyadenosine, CPT-11, cyclophosphamide, Docetaxel, doxorubicin, edotecarin, etoposide, fludarabine, 5-fluorouracil (5-FU), gemcitabine, HU-Gemzar, Irinotecan, methotrexate, 6-Mpurine, mytomicin-C, paclitaxel, cis-platin, SN-38, taxol, thiotepa, 6-thioguanine, trimetrexate vinblastine, vincristine, and VP-16.

24. The method of claim 23, wherein the anti-cancer agent is selected from the group consisting of gemcitabine, irinotecan, docetaxel, SN-38, carboplatin, doxorubicin and mytomicin C.

25. The method of claim 24, wherein the anti-cancer agent is gemcitabine.

26. The method of claim 24, wherein the anti-cancer agent is irinotecan.

27. The method of claim 24, wherein the anti-cancer agent is docetaxel.

28. The method of claim 17, wherein the anti-cancer agent is a DNA damaging agent.

29. The method of claim 28, wherein the DNA damaging agent is selected from alkylating agents, antimetabolites, antitumor antibiotics, platinum analogs, topoisomerase I inhibitors and topoisomerase II inhibitors.

30. The method of claim 17, wherein the anti-cancer agent is a mitotic inhibitor.

31. The method of claim 17, wherein at least one dose of (2R,Z)-2-amino-2-cyclohexyl-N-(5-(1-methyl-1H-pyrazol-4-yl)-1-oxo-2,6-dihydro-1H-[1,2]diazepino[4,5,6-cd]indol-8-yl)acetamide, a pharmaceutically acceptable salt or solvate thereof, or a mixture thereof, is administered 1 to 48 hours after the previous dose of anti-cancer treatment is administered.

32. The method of claim 17, wherein at least one dose of (2R,Z)-2-amino-2-cyclohexyl-N-(5-(1-methyl-1H-pyrazol-4-yl)-1-oxo-2,6-dihydro-1H-[1,2]diazepino[4,5,6-cd]indol-8-yl)acetamide, a pharmaceutically acceptable salt or solvate thereof, or a mixture thereof, is administered simultaneously with a dose of the anti-cancer treatment.
